(19) United States
(12) Patent Application Publication
(10) Pub. No.: US 2024/0186606 A1
(43) Pub. Date: Jun. 6, 2024

(54) SEPARATION RECOVERY METHOD OF METAL IONS, AND TWO-PHASE SEPARATED FLUID

(71) Applicant: FUJIFILM Corporation, Tokyo (JP)

(72) Inventors: Yo KUSHIDA, Kanagawa (JP); Hiroaki MOCHIZUKI, Kanagawa (JP)

(73) Assignee: FUJIFILM Corporation, Tokyo (JP)

(21) Appl. No.: 18/430,683

(22) Filed: Feb. 2, 2024

Related U.S. Application Data
(63) Continuation of application No. PCT/JP2022/035176, filed on Sep. 21, 2022.

(30) Foreign Application Priority Data
Sep. 29, 2021 (JP) 2021-159111

(51) Int. Cl. H01M 10/54; C22B 3/40

(52) U.S. Cl. CPC H01M 10/54; C22B 3/40

(57) ABSTRACT

Provided is a separation recovery method of metal ions and a two-phase separated fluid. The separation recovery method includes mixing a water phase including two or more kinds of metal ions and an organic compound with an oil phase including an extractant to move metal ions belonging to Group 8 to Group 12 from the water phase to the oil phase, the two or more kinds of metal ions including metal ions belonging to Group 3 to Group 16, and the organic compound coordinated to at least one kind of metal ions among the metal ions. In the two-phase separated fluid, a water phase including two or more kinds of metal ions including metal ions belonging to Group 3 to Group 16 and an organic compound and an oil phase including an extractant are phase-separated and present, and metal ions where the extractant is coordinate-bonded to metal elements belonging to Group 8 to Group 12 are present in the oil phase.

### CROSS-REFERENCE TO RELATED APPLICATIONS

This application is a Continuation of PCT International Application No. PCT/JP2022/035176 filed on Sep. 21, 2022, which claims priority under 35 U.S.C. § 119 (a) to Japanese Patent Application No. 2021-159111 filed in Japan on Sep. 29, 2021. Each of the above applications is hereby expressly incorporated by reference, in its entirety, into the present application.

### BACKGROUND OF THE INVENTION

### 1. Field of the Invention

The present invention relates to a separation recovery method of metal ions, and a two-phase separated fluid.

### 2. Description of the Related Art

Metals that can be mined from mines are limited, and stable supply of noble metals required for precision equipment is a big challenge. Accordingly, it is important to recover valuable metals from industrial waste irrespective of mining.

As a method of recycling waste into a metal, a wet extraction method (solvent extraction method) is used. In the wet extraction method, in a case where an aqueous solution (water phase) including ions of a metal element and an organic phase including a metal extractant are brought into contact with each other, mixed, and left to stand to separate the two phases, the ions of the metal element to which the metal extractant is coordinated can be moved (extracted) to the organic phase. By extracting the organic phase, stripping the ions of the metal element, and optionally purifying the ions, the waste can be recycled as a metal.

Incidentally, along with the recent spread of electric vehicles, the amount of lithium ion batteries (LiB) wasted has been increasing. In LiB, a positive electrode active material including a metal element such as cobalt or nickel is used, and a significant increase in the demand for cobalt or nickel is also expected along with the spread of electric vehicles or the like. In order to deal with the increase in the demand for valuable metals along with the tidal current, not only an increase in the amount of mining but also a technique of recycling wasted LiB into a metal are desired.

Therefore, a method of selectively separating and extracting cobalt and nickel is required, and a separation recovery method using a wet extraction method is considered.

For example, JP2020-105598A discloses “a method for recovering valuable metals, the method recovering at least cobalt among cobalt and nickel as valuable metals from an acidic solution obtained by performing a wet process on waste including a positive electrode material of a lithium ion secondary battery, the acidic solution including cobalt ions, nickel ions, and impurity, the method including: a first extraction step for Co recovery of extracting cobalt ions by solvent extraction from the acidic solution and stripping the cobalt ions; and a second extraction step for Co recovery of extracting cobalt ions by solvent extraction from a stripped solution obtained in the first extraction step for Co recovery and stripping the cobalt ions, in which the first extraction step for Co recovery includes: a solvent extraction process for extracting cobalt ions in the acidic solution into a solvent; a scrubbing process for scrubbing the solvent from which the cobalt ions are extracted; and a stripping process for stripping the cobalt ions in the solvent after the scrubbing into a solution”.

In addition, JP2019-179699A discloses “a method of manufacturing cobalt sulfate for a battery from electrolytic cobalt that is obtained by electrolysis and includes nickel, the method including: a dissolution step of dissolving the electrolytic cobalt in an acid; an extraction step of extracting and stripping cobalt ions in a cobalt solution obtained in the dissolution step by adding ammonia ions to the cobalt solution to adjust pH at the time of, before, or after contact between the cobalt solution and an extractant; and a crystallization step of crystallizing the cobalt ions in a stripped solution after the extraction step to obtain cobalt sulfate”.

### SUMMARY OF THE INVENTION

In the method described in JP2020-105598A, in the extraction step for Co recovery, the extractant is used alone or ammonia ions as a masking agent of nickel ions are used. In addition, as described above, cobalt ions are recovered through many steps. Therefore, from the viewpoint of productivity, a simpler recovery method is required. On the other hand, in the method described in JP2019-179699A, cobalt ions are extracted using ammonia ions as the masking agent of nickel ions and the extractant in combination. In this method, cobalt is recovered using electrolytic cobalt where the nickel content is typically relatively low at about 100 to 1000 mass ppm, and an application range thereof is limited. In addition, both of the methods described in JP2020-105598A and JP2019-179699A are methods of recovering cobalt and nickel and, in a case where other metals coexist, cannot be applied to a method of recovering the other metals. It is desirable to establish a recovery method that is not limited to these metals. An object of the present invention is to provide a separation recovery method of metal ions and a two-phase separated fluid, in which ions of metal elements belonging to Group 8 to Group 12 in the periodic table of elements can be separated and recovered with high separability (purity) even using a simple method from two or more kinds of metal ions including ions of metal elements belonging to Group 3 to Group 16 in the periodic table of elements.

The present inventors found that, in a wet extraction method of separating and recovering specific metal ions from two or more kinds of metal ions including ions of metal elements belonging to Group 3 to Group 16, through a simple operation of allowing a water phase including two or more kinds of metal ions to include an organic compound (B) having a coordinating functional group coordinated to at least one kind of metal ions among the two or more kinds of metal ions and bringing the water phase into contact with and mixing the water phase with an oil phase (organic phase) including an extractant (C), metal ions where the extractant (C) is coordinated to ions of metal elements belonging to Group 8 to Group 12 can be selectively moved and extracted to the oil phase.

The present invention has been completed as a result of repeated investigation based on the above findings.

That is, the above-described objects have been achieved by the following means.
<1> A separation recovery method of metal ions, the separation recovery method comprising:
mixing a water phase including two or more kinds of metal ions (A) and an organic compound (B) with an oil phase including an extractant (C) to move ions of metal elements belonging to Group 8 to Group 12 in a periodic table of elements from the water phase to the oil phase, the two or more kinds of metal ions (A) including ions of metal elements belonging to Group 3 to Group 16 in the periodic table of elements, and the organic compound (B) having a coordinating functional group coordinated to at least one kind of metal ions among the two or more kinds of metal ions (A).
<2> The separation recovery method according to <1>,
in which the extractant (C) is an acidic extractant.
<3> The separation recovery method according to <1> or <2>,
in which the extractant (C) is represented by Formula (I) or Formula (II).

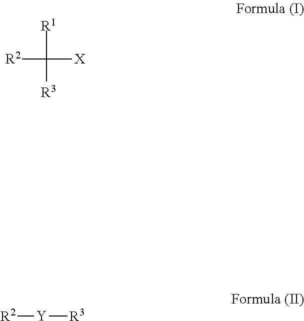

In Formula (I) and Formula (II), R¹ represents an alkyl group, R² and R³ represent an organic group and may be the same as or different from each other,

X represents a carboxy group, a sulfonate group, a sulfinate group, a phosphate group, a phosphonate group, or an oxime group, and

Y represents a phosphinate group, a phosphonate group, a phosphate group, a sulfonate group, or an oxime group.
<4> The separation recovery method according to any one of <1> to <3>,
in which the organic compound (B) is a chelating agent.
<5> The separation recovery method according to any one of <1> to <4>,
in which the organic compound (B) includes at least one element of N, O, S, or P in a molecular structure of the organic compound (B).
<6> The separation recovery method according to any one of <1> to <5>,
in which the organic compound (B) is represented by Formula (III).

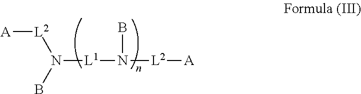

In Formula (III), L¹ represents a divalent organic group, L² represents a single bond or a divalent organic group, and two L²'s may be different from each other,

A represents a hydroxy group, an amino group, a carboxy group, a sulfonate group, a sulfinate group, a phosphate group, phosphonic acid, a cyano group, a carbamoyl group, or a thiol group, and two A's may be different from each other,

B represents a monovalent organic group or a hydrogen atom and, in a case where a plurality of B's are included, the plurality of B's may be different from each other, and

n represents an integer of 0 to 8.
<7> The separation recovery method according to any one of <1> to <6>,
in which the two or more kinds of metal ions (A) include at least one transition metal element.
<8> The separation recovery method according to any one of <1> to <7>,
in which the metal ions to be moved to the oil phase are ions of two metal elements belonging to different groups among ions of metal elements belonging to Group 8 to Group 11 in the periodic table of elements.
<9> The separation recovery method according to any one of <1> to <8>,
in which the two or more kinds of metal ions (A) are a metal recovery from a waste battery.
<10> A two-phase separated fluid,
in which a water phase including two or more kinds of metal ions (A) and an organic compound (B) and an oil phase including an extractant (C) are phase-separated and present in a state where the water phase and the oil phase are in contact with each other, the two or more kinds of metal ions (A) including ions of metal elements belonging to Group 3 to Group 16 in a periodic table of elements, and the organic compound (B) having a coordinating functional group coordinated to at least one kind of metal ions among the two or more kinds of metal ions (A), and
metal ions where the extractant (C) is coordinate-bonded to metal elements belonging to Group 8 to Group 12 in the periodic table of elements among the two or more kinds of metal ions (A) are present in the oil phase.
<11> The two-phase separated fluid according to <10>,
in which the extractant (C) is an acidic extractant.
<12> The two-phase separated fluid according to <10>or <11>,
in which the extractant (C) is represented by Formula (I) or Formula (II).

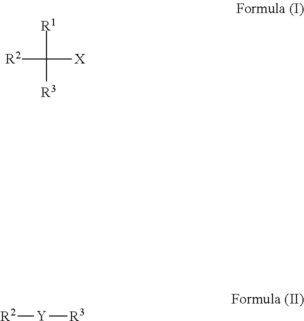

In Formula (I) and Formula (II), R¹ represents an alkyl group, R² and R³ represent an organic group and may be the same as or different from each other,

X represents a carboxy group, a sulfonate group, a sulfinate group, a phosphate group, a phosphonate group, or an oxime group, and

Y represents a phosphinate group, a phosphonate group, a phosphate group, a sulfonate group, or an oxime group.
<13> The two-phase separated fluid according to any one of <10> to <12>,
in which the organic compound (B) is a chelating agent.
<14> The two-phase separated fluid according to any one of <10> to <13>,
in which the organic compound (B) includes at least one element of N, O, S, or P in a molecular structure of the organic compound (B).
<15> The two-phase separated fluid according to any one of <10> to <14>,
in which the organic compound (B) is represented by Formula (III).

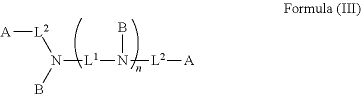

In Formula (III), L¹ represents a divalent organic group, L² represents a single bond or a divalent organic group, and two L²'s may be different from each other,

A represents a hydroxy group, an amino group, a carboxy group, a sulfonate group, a sulfinate group, a phosphate group, phosphonic acid, a cyano group, a carbamoyl group, or a thiol group, and two A's may be different from each other,

B represents a monovalent organic group or a hydrogen atom and, in a case where a plurality of B's are included, the plurality of B's may be different from each other, and n represents an integer of 0 to 8.
<16> The two-phase separated fluid according to any one of <10> to <15>,
in which the two or more kinds of metal ions (A) include at least one transition metal element.
<17> The two-phase separated fluid according to any one of <10> to <16>,
in which the metal ions present in the oil phase are ions of two metal elements belonging to different groups among ions of metal elements belonging to Group 8 to Group 11 in the periodic table of elements.

According to the present invention, it is possible to provide a separation recovery method of metal ions and a two-phase separated fluid, in which ions of metal elements belonging to Group 8 to Group 12 in the periodic table of elements can be separated and recovered with high separability (purity) even using a simple method from two or more kinds of metal ions including ions of metal elements belonging to Group 3 to Group 16 in the periodic table of elements.

The above-described and other characteristics and advantageous effects of the present invention will be clarified from the following description appropriately with reference to the accompanying drawings.

### DESCRIPTION OF THE PREFERRED EMBODIMENTS

In the present invention, numerical ranges represented by “to” include numerical values before and after “to” as lower limit values and upper limit values. In a case where a plurality of numerical ranges are set and described for the content, physical properties, and the like of a component in the present invention, the upper limit value and the lower limit value, which form each of the numerical ranges, are not limited to a specific combination described before and after “to” as a specific numerical range and can be set to a numerical range obtained by appropriately combining the upper limit value and the lower limit value of each numerical range.

In the present invention, the expression of a compound (for example, in a case where a compound is represented by an expression with “compound” added to the end) refers to not only the compound itself but also a salt or an ion thereof. In addition, this expression also refers to a derivative obtained by modifying a part of the compound, for example, by introducing a substituent into the compound within a range where the effects of the present invention do not deteriorate.

A substituent, a linking group, or the like (hereinafter, referred to as “substituent or the like”) is not specified in the present invention regarding whether to be substituted or unsubstituted may have an appropriate substituent. Accordingly, even in a case where a YYY group is simply described in the present invention, this YYY group includes not only an aspect not having a substituent but also an aspect having a substituent. The same shall be applied to a compound which is not specified in the present specification regarding whether to be substituted or unsubstituted. Examples of a preferable substituent include a group selected from the substituent Z described below.

In the present invention, in a case where a plurality of substituents or the like represented by a specific reference numeral are present or a plurality of substituents or the like are simultaneously or alternatively defined, the respective substituents or the like may be the same as or different from each other. In addition, unless specified otherwise, in a case where a plurality of substituents or the like are adjacent to each other, the substituents may be linked or fused to each other to form a ring.

In the present invention, unless specified otherwise, “ppm” representing a content or the like is based on mass and represents “mass ppm”.

### [Separation Recovery Method of Metal Ions]

A separation recovery method of metal ions according to an embodiment of the present invention (hereinafter, also referred to as the separation recovery method according to the embodiment of the present invention) includes mixing a water phase including two or more kinds of metal ions (A) and an organic compound (B) with an oil phase including an extractant (C) to move ions of metal elements belonging to Group 8 to Group 12 in a periodic table of elements from the water phase to the oil phase and to be present (remain) in the water phase for separation and recovery from the metal ions, the two or more kinds of metal ions (A) including ions of metal elements belonging to Group 3 to Group 16 in the periodic table of elements, and the organic compound (B) having a coordinating functional group coordinated to at least one kind of metal ions among the two or more kinds of metal ions (A). As a result, the ions of the metal elements belonging to Group 8 to Group 12 present in the water phase can be separated and recovered with high separability (high purity).

The details of the reason for this are not clear but considered to be as follows.

As the ions of the metal elements (simply referred to as the metal ions) present in the water phase, metal ions coordinated to the organic compound (B) and metal ions not coordinated to the organic compound (B) are considered to coexist. In the coexistence state, in a case where the water phase including the two or more kinds of metal ions and the oil phase including the extractant (C) are mixed, it is considered that, due to easy formation of a coordinate bond of the organic compound (B) and the extractant (C) to the metal ions, stability, and the like, the organic compound (B) coordinated to the metal ions is replaced with the extractant (C) or the extractant (C) is coordinated to the metal ions to which the organic compound (B) is not coordinated such that the metal ions to which the organic compound (B) is preferentially or selectively coordinated are selectively moved and extracted to the oil phase.

In the separation recovery method according to the embodiment of the present invention, easy coordination to the metal ions belonging to Group 3 to Group 16, in particular, the metal ions belonging to Group 8 to Group 12, stability, and the like can be controlled by selecting the organic compound (B) and the extractant (C) described below.

### <Water Phase>

In the separation recovery method according to the embodiment of the present invention, for the oil phase described below, the water phase including the two or more kinds of metal ions (A) and the organic compound (B) is used, the two or more kinds of metal ions (A) including ions of metal elements belonging to Group 3 to Group 16 in the periodic table of elements.

### (Two or More Kinds of Metal Ions (A))

The two or more kinds of metal ions (A) present in the water phase are two or more kinds of metal ions belonging to Group 3 to Group 16, and include at least one kind of metal ions belonging to Group 8 to Group 12 to be extracted, preferably, Group 8 to Group 11.

The two or more kinds of metal ions (A) only need to include at least one kind of metal ions belonging to Group 3 to Group 16, and may include metal ions belonging to groups other than Group 3 to Group 16. In the present invention, it is preferable that two or more kinds of metal ions belonging to Group 3 to Group 16 are included, it is more preferable that ions of at least one transition metal element (metal element belonging to Group 3 to Group 12), and it is still more preferable that two or more kinds of metal ions belonging to Group 6 to Group 12 are included. Note that at least one kind of metal ions among the two or more kinds of metal ions are metal ions belonging to Group 8 to Group 12 to be extracted, preferably, Group 8 to Group 11. As the metal ions (A), two or more kinds of metal ions belonging to Group 8 to Group 12 and preferably two or more kinds of metal ions belonging to Group 8 to Group 11 are included.

In addition, the number of kinds of the metal ions is not particularly limited as long as it is 2 or more. For example, the number of kinds of the metal ions can be 2 to 15 and is preferably 2 to 8 and more preferably 2 to 5.

A combination of the metal ions is not particularly limited, and examples of a combination of groups include a combination including Group 8 and Group 12, a combination including Group 9 and Group 10, a combination including Group 7 and Group 9, and a combination including Group 7 and Group 10. More specifically, for example, a combination of Group 8 and Group 12, a combination of Group 9 and Group 10, a combination of Group 7, Group 9, and Group 10, a combination of Group 9, Group 10, and Group 12, a combination of Group 8, Group 9, Group 10, and Group 12, or a combination further including Group 13 in addition to each of the combinations can be used.

In the present invention, the number of kinds of metal ions belonging to each of the groups may be two or more but, from the viewpoint of exhibiting high separability, is preferably one.

Specific examples of the combination of the metal ions include a combination including Co and Ni, a combination including Fe and Zn, a combination including Mn and Co, and a combination including Mn and Ni. More specifically, a combination of Fe and Zn, a combination of Co and Ni, a combination of Mn, Co, and Ni, a combination of Co, Ni, and Zn, a combination of Fe, Co, Ni, and Zn, or a combination further including In in addition to each of the combinations can be used.

The metal element belonging to each of the groups is not particularly limited, and an appropriate atom can be used.

Preferable examples of a metal element belonging to Group 3 include Sc and Y.

Preferable examples of a metal element belonging to Group 4 include Ti, Zr, and Hf.

Preferable examples of a metal element belonging to Group 5 include V, Nb, and Ta.

Preferable examples of a metal element belonging to Group 6 include Cr, Mo, and W.

Preferable examples of a metal element belonging to Group 7 include Mn and Tc.

Preferable examples of a metal element belonging to Group 8 include Fe, Ru, and Os.

Preferable examples of a metal element belonging to Group 9 include Co, Rh, and Ir.

Preferable examples of a metal element belonging to Group 10 include Ni, Pd, and Pt.

Preferable examples of a metal element belonging to Group 11 include Cu, Ag, and Au.

Preferable examples of a metal element belonging to Group 12 include Zn, Cd, and Hg.

Preferable examples of a metal element belonging to Group 13 include Al, Ga, In, and Tl.

Preferable examples of a metal element belonging to Group 14 include Ga, Sn, and Pb.

Preferable examples of a metal element belonging to Group 15 include Sb and Bi.

A metal element belonging to Group 16 is not particularly limited, and preferable examples thereof include Te.

Among the metal elements belonging to the respective groups, the metal element belonging to Period 4 or Period 5 is preferable.

The two or more kinds of metal ions (A) can be appropriately prepared and, for example, various metal salts (salts of typical elements with inorganic acids such as nitric acid or sulfuric acid or organic acids such as acetic acid), a mixture of mined metals (ion), a recovery from metal waste, other waste such as a metal recovery from a waste battery (LiB), or a mixture thereof can be used. Examples of the metal recovery from a wasted LiB include the recovery by the wet process described in JP2020-105598A and the electrolytic cobalt described in JP2019-179699A. A recovery method of the metal recovery can be appropriately found in the content of each of JP2020-105598A and JP2019-179699A, the content of which is incorporated as it is as a part of the description of the present specification.

### (Organic Compound (B))

The organic compound (B) is a compound having a coordinating functional group coordinated to at least one kind of metal ions belonging to Group 3 to Group 16. It is considered that the organic compound (B) is water-soluble, is present in the water phase, is coordinate-bonded to at least one kind of metal ions that coexist, and has a function of inhibiting the extractant (C) described below to form a coordinate bond such that the metal ions to which the organic compound (B) is coordinate-bonded remains in the water phase without being moved to the oil phase. That is, in the present invention, as the organic compound (B), a compound having a coordinating functional group coordinated to the metal ions that remain in the water phase without being moved to the oil phase is selected.

In the present invention, the water-soluble property refers to a property in which the organic compound (B) is soluble in water in a content described below.

The organic compound (B) is not particularly limited as long as it is a compound having the above-described function, and preferable examples thereof include a ligand having a coordinating functional group. The organic compound (B) may be a monodentate ligand but, from the viewpoint of separability, is preferably a multidentate ligand (chelating agent) and more preferably a bidentate to hexadentate ligand.

It is preferable that the organic compound (B) includes at least one element of N, O, S, or P in a molecular structure thereof, it is more preferable that the organic compound (B) includes the above-described element in a coordinating functional group, and it is still more preferable that the organic compound (B) includes the above-described element in all the coordinating functional groups. The number of the elements in the organic compound (B) is not particularly limited and, for example, can be 1 to 20 and is preferably 2 to 14 and more preferably 4 to 12. In a case where the organic compound (B) includes a plurality of the elements, the kinds of the elements may be the same as or different from each other. The coordinating functional group in the element is not particularly limited, and preferable examples thereof include a group that can be used as A in Formula (III) described below.

From the viewpoint that high separability can be realized with the extractant (C) described below, the organic compound (B) is more preferably a compound represented by Formula (III).

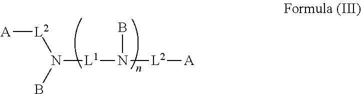

In Formula (III), L¹ represents a divalent organic group, and L² represents a single bond or a divalent organic group. The organic group that can be used as L¹ or L² is not particularly limited and is, for example, a group derived from an aromatic compound, an aliphatic compound, or a compound including a combination thereof. Specific examples of the organic group include an alkylene group (having preferably 1 to 12 carbon atoms, more preferably 1 to 6 carbon atoms, and still more preferably 1 to 3 carbon atoms), an alkenylene group (having preferably 2 to 6 carbon atoms and more preferably 2 or 3 carbon atoms), an arylene group (having preferably 6 to 24 carbon atoms and more preferably 6 to 10 carbon atoms), and a group relating to a combination thereof. The alkylene group and the alkenylene group may be linear, branched, or cyclic, and is preferably linear or branched and may include at least one, preferably two or more oxygen atoms, sulfur atoms, or nitrogen atoms in a carbon chain. The number of groups to be combined only needs to be 2 or more and is preferably 2 or 3. As the group relating to the combination, a combination of an alkylene group or an alkenylene group and an arylene group is preferable, and a combination of an alkylene group-an arylene group-an alkylene group is more preferable. As the organic group that can be used as L¹, an alkylene group is preferable, a linear alkylene group is more preferable, a linear group where N in Formula (III) is bonded to both ends of the longest carbon chain is still more preferable, and a 1,2-ethylene group is still more preferable. The number of carbon atoms in the alkylene group that can be used as LI is still more preferably 1 to 3 and still more preferably 2 in the above-described range. As the organic group that can be used as L², an alkylene group is preferable, a linear or branched alkylene group is more preferable, a linear alkylene group where N and A in Formula (III) is bonded to one end part of a carbon chain is still more preferable, a 1,1-linear alkanediyl group is still more preferable, and a methylene group is most preferable. The number of carbon atoms in the alkylene group that can be used as L² is still more preferably 1 to 3 and still more preferably 1 in the above-described range. L¹ and L² may be the same as or different from each other, and two L²'s in the formula may be different from each other.

A represents a hydroxy group, an amino group, a carboxy group, a sulfonate group (—SO₃H), a sulfinate group (—SO₂H), a phosphate group (—OPO₃H₂), a phosphonate group (—PO₃H₂), a cyano group, a carbamoyl group, or a thiol group. As A, a hydroxy group, a carboxy group, a phosphate group, or a phosphonate group is preferable. All of a sulfonate group, a sulfinate group, a phosphate group, and a phosphonate group include a group where at least one oxygen atom is substituted with a nitrogen atom or a sulfur atom.

Two A's may be the same as or different from each other.

A may be dissociated or may form a salt in the water phase depending on pH. A cation that forms a salt is not particularly limited, and examples thereof include a metal cation, in particular, a metal cation belonging to Group 1 or Group 2, and an organic cation. The organic cation is not particularly limited, and examples thereof include an ammonium cation and an alkylammonium cation.

Two -L₂-A groups in the compound represented by Formula (III) may be different from each other but are preferably the same as each other.

B represents a monovalent organic group or a hydrogen atom. The organic group that can be used as B is not particularly limited and is, for example, a group derived from an aromatic compound, an aliphatic compound, or a compound including a combination thereof or a -L2-A group. Specific examples of the organic group include an alkyl group (having preferably 1 to 12 carbon atoms, more preferably 1 to 6 carbon atoms, and still more preferably 1 to 3 carbon atoms), an alkenyl group (having preferably 2 to 6 carbon atoms and more preferably 2 or 3 carbon atoms), an aryl group (having preferably 6 to 24 carbon atoms and more preferably 6 to 10 carbon atoms), and a group relating to a combination thereof. The alkyl group and the alkenyl group may be linear, branched, or cyclic and are preferably linear or branched. The number of groups to be combined only needs to be 2 or more and is preferably 2 or 3.

Examples of the -L₂-A group that can be used as B include a group where L₂ and A are appropriately combined and a group where preferable examples of L₂ and A are combined. The -L₂-A group that can be used as B may be different from the -L₂-A group in Formula (III) but is preferably the same as the -L₂-A group in Formula (III). In a case where the compound represented by Formula (III) includes a plurality of B's, the plurality of B's may be the same as or different from each other. In addition, it is more preferable that two -L₂-A groups and a plurality of B's in the compound represented by Formula (III) are the same.

n represents an integer of 0 to 8, preferably an integer of 1 to 4, and more preferably 1 or 2.

The organic compound (B) may have a substituent, and preferable examples of the substituent which may be included include a group selected from a substituent Z described below, where the group does not correspond to A.

The organic compound (B) is preferably an amine compound (n=0) represented by Formula (III) or a mono or poly(alkylenediamine) compound represented by Formula (III) (n=1 to 8), and preferable examples of the mono or poly(alkylenediamine) compound include an alkylenediamine compound, a dialkylenetriamine compound, a trialkylenetetramine compound, a tetraalkylenepentamine compound, and a pentaalkylenehexamine compound.

Specific examples of the organic compound (B) include the following compounds in addition to compounds used in Examples, but the present invention is not limited thereto.

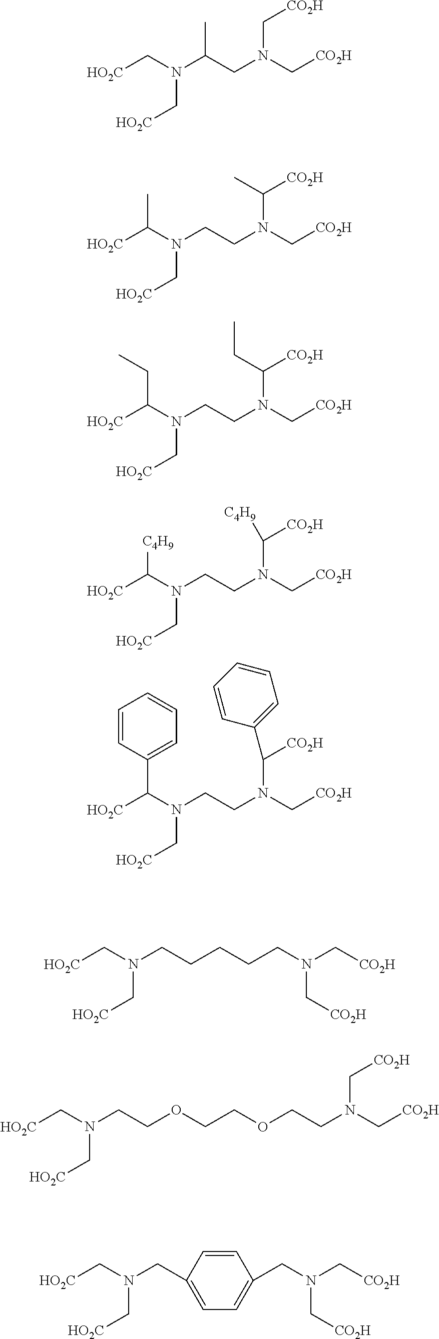

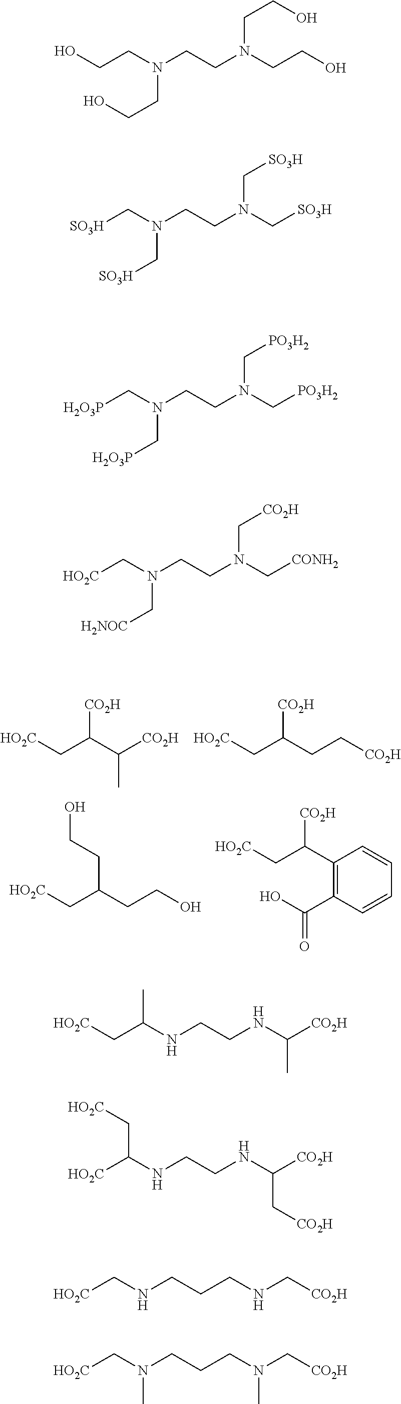

Water formed in the water phase is not particularly limited, and (super) pure water, ion exchange water, or the like can be used.

A total content of the two or more kinds of metal ions (A) in the water phase is not particularly limited and is appropriately set. For example, the total content can be 1,000 to 1,000,000 mass ppm and is preferably 1,000 to 100,000 mass ppm and more preferably 1,000 to 50,000 mass ppm.

A total content of the metal ions belonging to Group 8 to Group 12 among the metal ions is not particularly limited and is appropriately set. For example, the total content can be 1,000 to 80,000 mass ppm and is preferably 1,000 to 60,000 mass ppm and more preferably 1,000 to 30,000 mass ppm.

A total content of the metal ions belonging to Group 3 to Group 7 and Group 13 to Group 16 among the metal ions is not particularly limited and is appropriately set. For example, the total content can be 1,000 to 60,000 mass ppm and is preferably 1,000 to 30,000 mass ppm.

A content of the metal ions belonging to Group 8 among the metal ions is not particularly limited and is appropriately set. For example, the content can be 1,000 to 60,000 mass ppm and is preferably 1,000 to 20,000 mass ppm.

A content of the metal ions belonging to Group 9 among the metal ions is not particularly limited and is appropriately set. For example, the content can be 1,000 to 60,000 mass ppm and is preferably 1,000 to 20,000 mass ppm.

A content of the metal ions belonging to Group 10 among the metal ions is not particularly limited and is appropriately set. For example, the content can be 1,000 to 60,000 mass ppm and is preferably 1,000 to 20,000 mass ppm.

A content of the metal ions belonging to Group 11 among the metal ions is not particularly limited and is appropriately set. For example, the content can be 1,000 to 60,000 mass ppm and is preferably 1,000 to 20,000 mass ppm.

A content of the metal ions belonging to Group 12 among the metal ions is not particularly limited and is appropriately set. For example, the content can be 1,000 to 60,000 mass ppm and is preferably 1,000 to 20,000 mass ppm.

In a case where two or more kinds of metal ions belonging to each of the groups are present, the content of the metal ions belonging to each of the groups is the total content.

In the present invention, the content of the metal ions belonging to each of Group 8 to Group 12 may be more than or less than a content of the metal ions belonging to a specific group. In the separation recovery method according to the embodiment of the present invention, the metal ions can be separated and recovered with high separability. Therefore, the contents of metal ions belonging to different groups do not need to be set at a specific ratio. For example, the electrolytic cobalt described in JP2019-179699A includes nickel at a ratio of 100 to 1,000 mass ppm (0.01 to 0.1 mass %) with respect to cobalt. In the present invention, the content of the nickel can be set at the above-described ratio or more. This point is not limited to a combination of nickel and cobalt. For example, a mass ratio of the content of metal ions belonging to another group to the content of metal ions belonging to a specific group [the content of the metal ions belonging to the specific group:the content of the metal ions belonging to the other group] can be, for example, 100:1 to 10,000 and is preferably 100:10 to 5,000 and more preferably 100:50 to 1,000.

The content of the organic compound (B) in the water phase is appropriately set in consideration of the content of the metal ions, the amount of coordination to the metal ions, the number of the coordinating functional groups, and the like. For example, the content of the organic compound (B) with respect to 100 parts by mass of the total content of the metal ions can be 10 to 10,000 parts by mass and is preferably 40 to 5,000 parts by mass. On the other hand, the content of the organic compound (B) with respect to the total content of the metal ions to which the organic compound (B) can be coordinated (also referred to as the mixing amount; a ratio of the number of moles of the metal extractant to the total number of moles of the metal ions: molar ratio) can be, for example, 0.8 to 5.0 equivalents and is preferably 1.0 to 2.0 equivalents. Here, the metal ions to which the organic compound (B) can be coordinated refer to metal ions to which the organic compound (B) can be preferentially or selectively coordinated rather than the other metal ions in the water phase.

The pH of the water phase is not particularly limited and is appropriately set. In consideration of the solubility of the metal ions, the formation of complex ions, and the like, the pH of the water phase is, for example, preferably 0.1 to 10 and more preferably 0.5 to 7.

The temperature of the water phase is not particularly limited and can be, for example, 10° C. to 60° C.

The water phase can be prepared by dissolving the metal ions and the organic compound (B) in water. It is preferable that the water phase is prepared by mixing an aqueous solution in which the metal ions are dissolved and an aqueous solution in which the organic compound (B) is dissolved with each other. In this case, in order to coordinate-bond the organic compound (B) to at least one kind of metal ions, it is preferable to mix both of the aqueous solutions at the following preparation temperature at pH of 0.1 to 10 for 10 minutes to 6 hours. In this case, in order to dissolve the organic compound (B) in water or to adjust the pH of the water phase, an acid or an alkali can also be used. As the acid, a well-known acid can be used without any particular limitation, and examples thereof include an inorganic acid such as sulfuric acid, hydrochloric acid, nitric acid, or phosphoric acid and an organic acid such as formic acid, acetic acid, oxalic acid, organic phosphoric acid, or organic sulfonic acid. As the alkali, a well-known alkali can be used without any particular limitation, and examples thereof include an inorganic alkali and an organic alkali. Among these, an inorganic alkali is preferable. Examples of the inorganic alkali include a hydroxide of a metal belonging to Group 1 or Group 2, a metal alkali such as a carbonate, ammonia water, and ammonium chloride. Examples of the organic alkali include an organic ammonium salt. The amount of the acid or the alkali agent used is not particularly limited and, for example, can be 0.25 to 1.75 molar equivalents and is preferably 0.5 to 1.5 molar equivalents with respect to the coordinating functional group in the organic compound (B).

Preparation conditions of the water phase are not particularly limited. For example, the preparation temperature can be 10° C. to 60° C.

### <Oil Phase>

In the separation recovery method according to the embodiment of the present invention, the oil phase (organic phase) including the extractant (C) is used for the above-described water phase.

### (Extractant (C))

The extractant (C) is a compound having a coordinating functional group coordinated to metal ions belonging to Group 8 to Group 12. The extractant (C) exhibits solubility in an organic solvent, is present in the oil phase, is coordinate-bonded to metal ions present in the vicinity of an interface between the water phase and the oil phase, and has a function of moving the metal ions to the oil phase.

In the present invention, the solubility in the organic solvent refers to a property in which the extractant (C) is soluble in the organic solvent in a content described below.

The extractant (C) is not particularly limited as long as it is a compound having the above-described function, and preferable examples thereof include a ligand having a coordinating functional group. The extractant (C) may be a monodentate ligand or a multidentate ligand (chelating agent). In a case where the extractant (C) is a multidentate ligand, the extractant (C) is preferably a bidentate to octadentate ligand.

The extractant (C) is preferably an acidic extractant. In the present invention, the acidic extractant refers to a compound having an acidic functional group that dissociates hydrogen ions (H⁺) in a molecular structure thereof, and specifically can be defined by an acid dissociation constant pKa. The pKa of the extractant (C) is, for example, preferably 1 to 12 and more preferably 2 to 8. In the present invention, the pKa is a value measured by neutralization titration.

From the viewpoint that high separability can be realized with the above-described organic compound (B), the extractant (C) is more preferably a compound represented by Formula (I) or Formula (II).

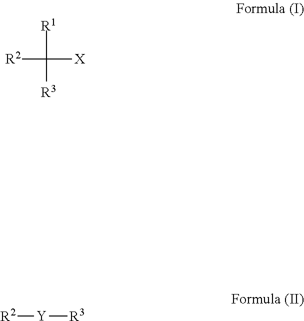

In Formula (I), R¹ represents an alkyl group. The alkyl group may be linear, branched, or cyclic, and the number of carbon atoms in the alkyl group is not particularly limited. The number of carbon atoms in the alkyl group that can be used as R¹ is not particularly limited and, for example, is preferably 1 to 20, more preferably 1 to 6, and still more preferably 1 to 3.

In Formula (I) and Formula (II), R² and R³ represent an organic group. The organic group that can be used as R² and R³ is not particularly limited and is, for example, a group derived from an aromatic compound, an aliphatic compound, or a compound including a combination thereof. Specific examples of the organic group include an alkyl group (having preferably 1 to 20 carbon atoms, more preferably 4 to 16 carbon atoms, and still more preferably 6 to 12 carbon atoms), an alkenyl group (having preferably 1 to 20 carbon atoms and more preferably 4 to 16 carbon atoms), an aryl group (having preferably 6 to 24 carbon atoms and more preferably 6 to 10 carbon atoms), and a group relating to a combination thereof. The alkyl group and the alkenyl group may be linear, branched, or cyclic. The number of groups to be combined only needs to be 2 or more and is preferably 2 or 3. Among the above-described examples, an alkyl group is preferable as the organic group that can be used as R² and R³. The divalent organic groups that can be used as R² and R³ may be the same as or different from each other.

In the compound represented by Formula (I), it is preferable that all of R¹ to R³ represent an alkyl group, and it is more preferable that one of R¹ to R³ represents a long chain alkyl group having 4 to 16 carbon atoms and the remaining two of R¹ to R³ represent a single chain alkyl group having 1 to 3 carbon atoms. In the compound represented by Formula (II), it is preferable that both of R² and R³ represent an alkyl group, it is more preferable that both of R² and R³ represent a long chain alkyl group having 4 to 12 carbon atoms, and it is still more preferable that both of R² and R³ represent the same alkyl group.

In Formula (I), X represents a carboxy group, a sulfonate group (—SO₃H), a sulfinate group (—SO₂H), a phosphate group (—OPO₃H₂), a phosphonate group (—PO₃H₂), or an oxime group (—CH═N—OH, —CR═N—OH, where R represents an organic group). All of a phosphonate group, a sulfinate group, a phosphate group, and a phosphonate group include a group where at least one oxygen atom is substituted with a nitrogen atom or a sulfur atom.

In Formula (II), Y represents a phosphinate group (—P(═O)(OH)—), a phosphonate group (—P(═O)(OH)O—), a phosphate group (—OP(═O)(OH)O—), a sulfonate group (—S(═O)₂O—), or an oxime group (>C═N—OH). All of a phosphinate group, a phosphonate group, a phosphate group, and a sulfonate group include a group where at least one oxygen atom is substituted with a nitrogen atom or a sulfur atom.

X and Y may be dissociated or may form a salt in the oil phase. A cation that forms a salt is not particularly limited, and examples thereof include a metal cation and an organic cation.

Among the above-described compounds, a phosphonate compound (compound represented by Formula (II) having a phosphonate group as Y) is preferable as the extractant (C).

The extractant (C) may have a substituent, and preferable examples of the substituent which may be included include a group selected from a substituent Z described below, where the group does not correspond to X.

Specific examples of the extractant (C) include the following compounds in addition to compounds used in Examples, but the present invention is not limited thereto.

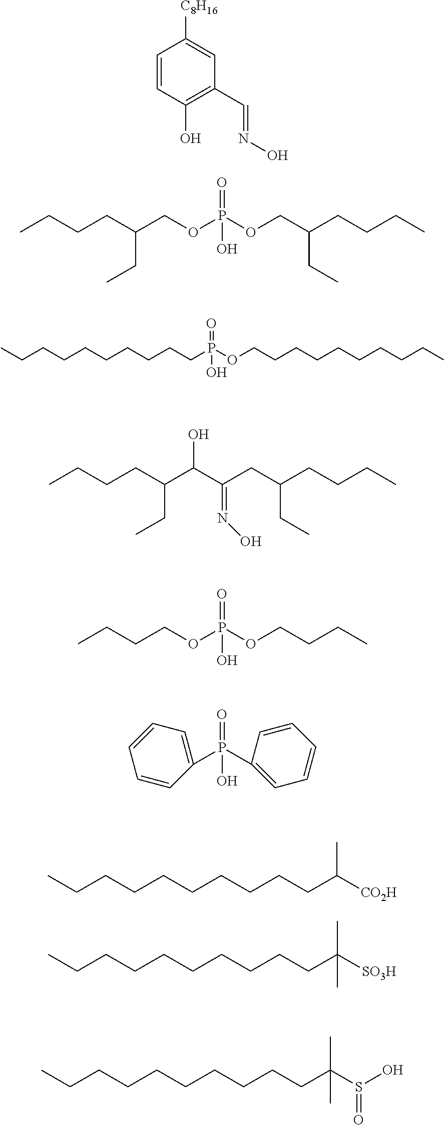

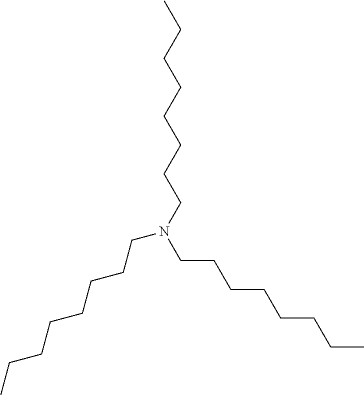 [SMILES: CCCCCCCCN(CCCCCCCC)CCCCCCCC]

A combination of the organic compound (B) and the extractant (C) that is suitably used for the metal ions to be extracted is not uniquely determined and is appropriately determined in consideration of the number of metal ions to be coordinated, a complex formation constant of the metal ions and the extractant (C) or the organic compound (B), pH during mixing, the coordinating functional group of the organic compound (B), the pKa of the extractant (C), and the like.

Examples of the substituent which may be included in the organic compound (B) and the extractant (C) include the following substituent Z.

### —Substituent Z—

The substituent Z includes: an alkyl group (preferably an alkyl group having 1 to 20 carbon atoms, for example, methyl, ethyl, isopropyl, t-butyl, pentyl, heptyl, 1-ethylheptyl, benzyl, 2-ethoxyethyl, or 1-carboxymethyl); an alkenyl group (preferably an alkenyl group having 2 to 20 carbon atoms, for example, vinyl, allyl, or oleyl); an alkynyl group (preferably an alkynyl group having 2 to 20 carbon atoms, for example, ethynyl, butadiynyl, or phenyl-ethynyl); a cycloalkyl group (preferably a cycloalkyl group having 3 to 20 carbon atoms; for example, cyclopropyl, cyclopentyl, cyclohexyl, or 4-methylcyclohexyl; the meaning of an alkyl group described in the present invention typically includes a cycloalkyl group but, here, an alkyl group and a cycloalkyl group are distinguished from each other); an aryl group (preferably an aryl group having 6 to 26 carbon atoms, for example, phenyl, 1-naphthyl, 4-methoxyphenyl, 2-chlorophenyl, or 3-methylphenyl); an aralkyl group (preferably an aralkyl group having 7 to 23 carbon atoms, for example, benzyl or phenethyl); a heterocyclic group (preferably a heterocyclic group having 2 to 20 carbon atoms and more preferably a 5- or 6-membered heterocyclic group having at least one oxygen atom, sulfur atom, or nitrogen atom; the heterocyclic group includes an aromatic heterocyclic group and an aliphatic heterocyclic group; for example, a tetrahydropyran ring group, a tetrahydrofuran ring group, 2-pyridyl, 4-pyridyl, 2-imidazolyl, 2-benzimidazolyl, 2-thiazolyl, 2-oxazolyl, a pyrrolidone group); an alkoxy group (preferably an alkoxy group having 1 to 20 carbon atoms, for example, methoxy, ethoxy, isopropyloxy, or benzyloxy); an aryloxy group (preferably an aryloxy group having 6 to 26 carbon atoms, for example, phenoxy, 1-naphthyloxy, 3-methylphenoxy, or 4-methoxyphenoxy); a heterocyclic oxy group (a group in which an —O— group is bonded to the above-described heterocyclic group); an alkoxycarbonyl group (preferably an alkoxycarbonyl group having 2 to 20 carbon atoms, for example, ethoxycarbonyl, 2-ethylhexyloxycarbonyl, or dodecyloxycarbonyl); an aryloxycarbonyl group (preferably an aryloxycarbonyl group having 6 to 26 carbon atoms, for example, phenoxycarbonyl, 1-naphthyloxycarbonyl, 3-methylphenoxycarbonyl, or 4-methoxyphenoxycarbonyl); a heterocyclic oxycarbonyl group (a group in which an —O—CO— group is bonded to the above-described heterocyclic group); an amino group (preferably an amino group having 0 to 20 carbon atoms, an alkylamino group, or an arylamino group, for example, amino (—NH₂), N,N-dimethylamino, N,N-diethylamino, N-ethylamino, or anilino); a sulfamoyl group (preferably a sulfamoyl group having 0 to 20 carbon atoms, for example, N,N-dimethylsulfamoyl or N-phenylsufamoyl); an acyl group (an alkylcarbonyl group, an alkenylcarbonyl group, an alkynylcarbonyl group, an arylcarbonyl group, or a heterocyclic carbonyl group, preferably an acyl group having 1 to 20 carbon atoms, for example, acetyl, propionyl, butyryl, octanoyl, hexadecanoyl, acryloyl, methacryloyl, crotonoyl, benzoyl, naphthoyl, or nicotinoyl); an acyloxy group (an alkylcarbonyloxy group, an alkenylcarbonyloxy group, an alkynylcarbonyloxy group, or a heterocyclic carbonyloxy group, preferably an acyloxy group having 1 to 20 carbon atoms, for example, acetyloxy, propionyloxy, butyryloxy, octanoyloxy, hexadecanoyloxy, acryloyloxy, methacryloyloxy, crotonoyloxy, or nicotinoyloxy); an aryloyloxy group (preferably an aryloyloxy group having 7 to 23 carbon atoms, for example, benzoyloxy or naphthoyloxy); a carbamoyl group (preferably a carbamoyl group having 1 to 20 carbon atoms, for example, N,N-dimethylcarbamoyl or N-phenylcarbamoyl); an acylamino group (preferably an acylamino group having 1 to 20 carbon atoms, for example, acetylamino or benzoylamino); an alkylthio group (preferably an alkylthio group having 1 to 20 carbon atoms, for example, methylthio, ethylthio, isopropylthio, or benzylthio); an arylthio group (preferably an arylthio group having 6 to 26 carbon atoms, for example, phenylthio, 1-naphthylthio, 3-methylphenylthio, or 4-methoxyphenylthio); a heterocyclic thio group (a group in which an —S— group is bonded to the above-described heterocyclic group); an alkylsulfonyl group (preferably an alkylsulfonyl group having 1 to 20 carbon atoms, for example, methylsulfonyl or ethylsulfonyl); an arylsulfonyl group (preferably an arylsulfonyl group having 6 to 22 carbon atoms, for example, benzenesulfonyl); an alkylsilyl group (preferably an alkylsilyl group having 1 to 20 carbon atoms, for example, monomethylsilyl, dimethylsilyl, trimethylsilyl, or triethylsilyl); an arylsilyl group (preferably an arylsilyl group having 6 to 42 carbon atoms, for example, triphenylsilyl); an alkoxysilyl group (preferably an alkoxysilyl group having 1 to 20 carbon atoms, for example, monomethoxysilyl, dimethoxysilyl, trimethoxysilyl, or tricthoxysilyl); an aryloxysilyl group (preferably an aryloxysilyl group having 6 to 42 carbon atoms, for example, triphenyloxysilyl); a phosphoryl group (preferably a phosphate group having 0 to 20 carbon atoms, for example, —OP(═O)(R^{P})₂); a phosphonyl group (preferably a phosphonyl group having 0 to 20 carbon atoms, for example, —P(═O)(R^{P})₂); a phosphinyl group (preferably a phosphinyl group having 0 to 20 carbon atoms, for example, —P(R^{P})₂); a phosphonate group (preferably a phosphonate group having 0 to 20 carbon atoms, for example, —PO(OR^{P})₂); a sulfo group (sulfonate group); a carboxy group; a hydroxy group; a sulfanyl group; a cyano group; and a halogen atom (for example, a fluorine atom, a chlorine atom, a bromine atom, or an iodine atom). R^{P} represents a hydrogen atom or a substituent (preferably a group selected from the substituent Z).

In addition, each group exemplified in the substituent Z may be further substituted with the substituent Z.

The alkyl group, the alkylene group, the alkenyl group, the alkenylene group, the alkynyl group, the alkynylene group, and/or the like may be cyclic or chained, may be linear or branched.

### (Organic Solvent)

The organic solvent for forming the oil phase is not particularly limited, and an appropriate organic solvent can be used. Examples of the organic solvent include an alcohol solvent, an ether solvent, a hydrocarbon-based solvent (an aromatic solvent or an aliphatic solvent), and a halogen solvent. In particular, a hydrocarbon-based solvent is preferable, various solvents as components separated from petroleum are more preferable, and hydrocarbon-based solvents of aromatic groups, paraffin, naphthene, kerosine, gasoline, naphtha, heating oil, and light oil are still more preferable.

The content of the extractant (C) in the oil phase is appropriately set in consideration of the content of the metal ions, the amount of coordination to the metal ions, the number of the coordinating functional groups, and the like. For example, the content in the oil phase can be 20 to 10,000 millimole/L (mM), and is preferably 50 to 1,000 millimole/L.

The temperature of the oil phase is not particularly limited and can be, for example, 10° C. to 60° C.

The oil phase can be prepared by dissolving the extractant (C) in the organic solvent. Preparation conditions of the oil phase are not particularly limited. For example, the preparation temperature can be 10° C. to 60° C.

### Contact and Mixing>

In the separation recovery method according to the embodiment of the present invention, the water phase and the oil phase described above are mixed and left to stand.

In this case, mixing conditions and standing conditions are not particularly limited and can be appropriately set. For example, mixing can be performed using various mixing devices. Examples of a method using the mixing device include a method using a magnetic stirrer (stirrer chip), a method using a mechanical stirrer, and a method using a mixer. Stirring conditions (a stirring rate, a stirring time, and the like) only need to be conditions (conditions where the extractant (C) is coordinate-bonded to the metal ions) where the water phase and the oil phase can be mixed, and are appropriately set depending on the metal ions, the combination of the organic compound (B) and the extractant (C), and the mixing temperature, and the mixing device. For example, the stirring time is not uniquely determined depending on the stirring conditions and the like, and can be, for example, 10 minutes to 24 hours.

The standing conditions only need to be conditions where the water phase and the oil phase are separated into two layers. For example, the standing time can be 10 minutes to 24 hours after stopping mixing.

The mixing temperature and the standing temperature are not particularly limited and can be, for example, 10° C. to 60° C.

During the mixing of the water phase and the oil phase, a mixing ratio between the water phase and the oil phase is appropriately set depending on a metal ion concentration, a concentration of the organic compound (B), a concentration of the extractant (C), and the like, and is not uniquely determined. For example, in a case where the water phase and the oil phase that satisfy the respective concentrations are mixed, the ratio of the oil phase to 100 mL of the water phase can be 50 to 2,000 mL and is preferably 80 to 1,000 mL. On the other hand, focusing on the metal ions present in the water phase, it is preferable that the oil phase is mixed at a ratio of 1 to 20 moles of the extractant (C) to the total content (moles) of the metal ions, and it is more preferable that the oil phase is mixed at a ratio of 1 to 10 moles of the extractant (C) to the total content (moles) of the metal ions. In addition, the content of the extractant (C) with respect to the total content of the metal ions to which the extractant (C) can be coordinated (also referred to as the mixing amount; a ratio of the number of moles of the metal extractant to the total number of moles of the metal ions: molar ratio) can be, for example, 1.0 to 10.0 equivalents and is preferably 1.5 to 6.0 equivalents. Here, the metal ions that can be coordinated to the extractant (C) refers to metal ions that are coordinated to the extractant (C) and are extracted to the oil phase.

During the mixing of the water phase and the oil phase, the pH of the mixing system can be adjusted. Here, the pH that is set for specific metal ions to be extracted is not uniquely determined and is appropriately determined in consideration of the pKa of the metal extractant, the complex formation constants of the metal extractant and the metal ions, the number of metal ions to be coordinated, and the like. The pH of the mixing system can be, for example, 0.1 to 14 and is preferably 2 to 14 and more preferably 3 to 10. The adjustment of the pH can be performed using the acid or the alkali described above, an aqueous solution thereof, or the like, and one preferable aspect is an aspect where ammonium ions are not used.

In a case where the pH of the mixing system is adjusted during the mixing of the water phase and the oil phase, the mixing of the water phase and the oil phase and the standing after the mixing are performed after adjusting the pH.

In a two-phase separated fluid (a solvent extraction phase or a solvent extraction system) where the water phase and the oil phase are phase-separated that is obtained by mixing the water phase and the oil phase and leaving the mixture to stand, the water phase and the oil phase are phase-separated into layers and present in a state where they are in contact with each other. Metal ions where the extractant (C) is coordinate-bonded to metal elements belonging to Group 8 to Group 12, preferably, Group 8 to Group 11 among the two or more kinds of metal ions are present in (moved to) the oil phase, and two kinds of metal ions belonging to different groups to which the extractant (C) is coordinate-bonded among metal ions belonging to Group 8 to Group 12, preferably, Group 8 to Group 11 are also present in the oil phase. For example, the combination of the metal ions to be moved to the oil phase is a combination including two or more kinds of metal ions belonging to Group 8 to Group 12, preferably, Group 8 to Group 11 among the above-described combination of the metal ions, and particularly a combination of Co and Ni is preferable.

The metal ions to be extracted to the oil phase are not particularly limited as long as they are all the kinds in the water phase, may be one kind or two or more kinds, and are preferably one kind or two kinds. In a case where two kinds of metal ions are extracted, it is preferable that a concentration of one kind of metal ions is higher than a concentration of another kind of metal ions, for example, it is preferable that a concentration (by mass) of one kind of metal ions is two or more times with respect to a concentration of another kind of metal ions (the concentration of the other kind of metal ions is 50 mass % or less with respect to the concentration of the one kind of metal ions).

In the water phase forming the two-phase separated fluid, the metal ions (A) and the organic compound (B) may be present separately from each other, and the organic compound (B) may be present to be coordinated to the metal ions (A).

In the separation recovery method according to the embodiment of the present invention, using a simple method of mixing the water phase and the oil phase with each other and leaving the mixture to stand, one kind or two or more kinds of metal ions belonging to Group 8 to Group 12 among the two or more kinds of metal ions belonging to Group 3 to Group 16 can be separated, recovered, and extracted with high separability.

In a case where two or more kinds of metal ions belonging to Group 3 to Group 16 coexist, metal ions to be selectively extracted are not uniquely determined depending on the group, the period, or the content of the metal ions, the kind of the organic compound (B), the kind of the extractant (C), and the like.

Note that, in a case where metal ions belonging to Group 8 to Group 12 and metal ions belonging to Group 3 to Group 7 or Group 13 to Group 16 are present in the water phase, by using the organic compound (B) and the extractant (C), metal ions belonging to Group 8 to Group 12 tend to be selectively extracted from metal ions belonging to groups other than Group 8 to Group 12, and one kind or two or more kinds of metal ions belonging to Group 8 to Group 12 tend to be selectively extracted from the other metal ions. On the other hand, in a case where metal ions belonging to Group 3 to Group 7 or Group 13 to Group 16 are not present in the water phase, one kind or two or more kinds of metal ions belonging to any group among the metal ions belonging to Group 8 to Group 12 are selectively extracted. In either case, particularly in a case where metal ions belonging to Group 8 and Group 12 are Fe, Co, Ni, and Zn, respectively, the metal ions can be selectively extracted and separated with higher separability.

Particularly in a case where metal ions belonging to Group 8 and Group 12 are Fe and Zn, respectively, one of Fe or Zn is selectively extracted and separated from metal ions belonging to the other groups with higher separability. In addition, in a case where Fe is selectively extracted in the coexistence of Fe and Zn, Fe can be selectively extracted with high separability by using EDTA or EDA as the organic compound (B) and using phosphonic acid as the extractant (C). On the other hand, in a case where Zn is selectively extracted, Zn can be selectively extracted with high separability by using thiourea as the organic compound (B) and using TEHA as the extractant (C). During the selective extraction of Fe or Zn, it is preferable to set pH to 0.2 to 3.5.

In a case where metal ions belonging to Group 8 and Group 12 are not present in the water phase, among the above-described metal ions, metal ions belonging to Group 9 and Group 10 tend to be selectively extracted from metal ions belonging to the other groups, and one kind or two or more kinds of metal ions belonging to Group 9 and Group 10 tend to be selectively extracted from the other metal ions. Particularly in a case where metal ions belonging to Group 9 and Group 10 are Co and Ni, respectively, the metal ions can be selectively extracted and separated with higher separability. In addition, in a case where Co is selectively extracted in the coexistence of Co and Ni, Co can be selectively extracted with high separability by using EDTA-OH, DPTA, or EDTA as the organic compound (B) and using phosphonic acid as the extractant (C). On the other hand, in a case where Ni is selectively extracted, Ni can be selectively extracted with high separability by using EDDA as the organic compound (B) and using phosphonic acid as the extractant (C). During the selective extraction of Co or Ni, it is preferable to set pH to 3.6 to 8.5.

In the separation recovery method according to the embodiment of the present invention, as described above, ions of metal elements belonging to Group 8 to Group 12 can be selectively extracted and recovered with high separability to the oil phase from the two or more kinds of metal ions present in the water phase. Further, even in a case where two or more kinds of metal ions belonging to Group 8 to Group 12 are extracted, one kind of metal ions can be selectively extracted with a concentration that is two or more times than that of the other kinds of metal ions.

The separation recovery method according to the embodiment of the present invention can also be called an extraction method of metal ions.

The separation recovery method according to the embodiment of the present invention may include steps other than the step of mixing the water phase and the oil phase with each other and leaving the mixture to stand. Examples of the other steps include a method of stripping (isolating) metal ions from the oil phase obtained in the step of mixing the water phase and the oil phase with each other and leaving the mixture to stand, a step of recovering the stripped metal ions as a compound (salt), a step of purifying the stripped metal ions or the compound thereof, and a step of removing ions of metal elements belonging to Group 1 or Group 2 in the periodic table of elements in advance. As a method of stripping (isolating) the metal ions from the oil phase, a well-known method can be applied without any particular limitation. For example, the metal ions can be stripped by adjusting the liquid phase to be acidic, for example, pH of 2 to 4 using an inorganic acid such as sulfuric acid, hydrochloric acid, or nitric acid. For example, the details can be appropriately found in the content described in each of JP2020-105598A and JP2019-179699A, the content of which is incorporated as it is as a part of the description of the present specification. As a method of recovering the stripped metal ions as a compound, a well-known method can be applied without any particular limitation. For example, the details can be appropriately found in the content described in each of JP2020-105598A and JP2019-179699A, the content of which is incorporated as it is as a part of the description of the present specification.

### EXAMPLES

Hereinafter, the present invention will be described in more detail based on Examples but is not limited to these examples. “Parts” and “%” that represent compositions in the following examples are mass-based unless particularly otherwise described.

### Preparation of Metal Ion-Containing Aqueous Solution

15.5 g of iron (II) sulfate heptahydrate (manufactured by FUJIFILM Wako Pure Chemicals Corporation), 15.5 g of zinc (II) sulfate heptahydrate (manufactured by FUJIFILM Wako Pure Chemicals Corporation), 15.5 g of cobalt (II) sulfate heptahydrate (manufactured by FUJIFILM Wako Pure Chemicals Corporation), 15.5 g of nickel (II) sulfate heptahydrate (manufactured by FUJIFILM Wako Pure Chemicals Corporation), and 15.5 g of indium (III) sulfate (manufactured by Kanto Kagaku Co., Inc.) were added to a 1 L measuring flask, were diluted with ultrapure water, and were stirred and dissolved at 30° C. As a result, a metal ion-containing aqueous solution including five kinds of metal ions was prepared.

In addition, each of sulfates of a combination of metal ions shown in Table 1 was dissolved in ultrapure water to prepare each of metal ion-containing aqueous solutions including two kinds to four kinds of metal ions.

### <Preparation of Extractant Solution>

11.0 g of tris(2-ethylhexyl)amine (TEHA, manufactured by Tokyo Chemical Industry Co., Ltd.) as the extractant (C) was added to a 100 mL measuring flask, and was diluted using kerosine (manufactured by FUJIFILM Wako Pure Chemicals Corporation) at room temperature. As a result, a TEHA solution (concentration: 310 mM) was prepared as an extractant solution.

Each of extractant solutions having a concentration of 310 mM was prepared using the same method as that of the preparation of the TEHA solution, except that a compound shown in the column “Extractant (C)” of Table 1 was used instead of TEHA.

Regarding each of the compounds used as the extractant (C), pKa calculated using the above-described method is shown in Table 1.

### <Preparation of Organic Compound Aqueous Solution>

Thiourea (manufactured by FUJIFILM Wako Pure Chemicals Corporation) or ethylenediamine (EDA, manufactured by Tokyo Chemical Industry Co., Ltd.) as the organic compound (B) was dissolved in ultrapure water such that the organic compound concentration was 1 M (mol/L). As a result, each of organic compound aqueous solutions was prepared. In addition, EDTA, EDTA-OH, DPTA, EDDA, or NTA was added to ultrapure water such that the organic compound concentration was 1 M, sodium hydroxide equivalent to a molar concentration of a carboxyl group was added thereto for neutralization, and each of organic compounds was dissolved. As a result, each of the organic compound aqueous solutions was prepared.

### Example 1

### <Separation Recovery of Metal Ions>

1.5 mL of an aqueous solution in which thiourea as an organic compound aqueous solution was dissolved with respect to 10 mL of the prepared metal ion-containing aqueous solution was added to a 30 mL vial tube and was stirred at 25° C. for 10 minutes. This way, a water phase including 5 kinds of metal ions (A) and an organic compound (B) was prepared. In this case, a mixing amount (unit: equivalent) of the organic compound (B) with respect to a total content of the metal ions (here, Fe and Zn) that can be coordinated is shown in the column “Mixing Amount” of Table 1. The pH of the water phase was 4.1.

12 mL of a TEHA solution as the extractant solution was added to the water phase and was stirred using a stirrer chip at 25° C. for 30 minutes. In this case, a mixing amount (unit: equivalent) of the extractant (C) with respect to a total content of the metal ions (here, Fe and Zn) that can be coordinated is shown in the column “Mixing Amount” of Table 1. Next, a 10 M sodium hydroxide aqueous solution or a 10 M hydrochloric acid was added to adjust the pH of the mixed solution to a value shown in the column “pH during Mixing” of Table 1. Further, the solution was stirred at 25° C. for 30 minutes, and was left to stand at the same temperature for 1 hour. After verifying that the solution was separated into two layers of the organic phase (oil phase) and the water phase, the separated water phase was extracted, and the separation and recovery of the metal ions was performed.

### Examples 2 to 14

The separation and recovery of the metal ions according to Examples 2 to 14 was performed using the same method as that of Example 1, except that the metal ion-containing aqueous solution, the extractant solution, and the organic compound aqueous solution were changed to a combination shown in Table 1, the mixing amounts of the organic compound (B) and the extractant (C) were set to values shown in the columns “Mixing Amount” of Table 1, and the pH during the mixing of the water phase and the oil phase was set to a value shown in the column “pH during Mixing” of Table 1 for mixing and standing.

Here, “the metal ions that can be coordinated” is as described above and refers to metal ions shown in the column “Extracted Metal Ions” of Table 1 in each of the examples (hereinafter, the same can be applied).

### Example 15

### <Separation Recovery of Metal Ions>

1.5 mL of an aqueous solution in which EDTA as an organic compound aqueous solution was dissolved with respect to 10 mL of a metal ion-containing aqueous solution including metal ions shown in Table 1 was added to a 30 mL vial tube and was stirred at 25° C. for 10 minutes. This way, a water phase including 2 kinds of metal ions (A) and an organic compound (B) was prepared. In this case, a mixing amount (unit: equivalent) of the organic compound (B) with respect to a total content of the metal ions (here, Co and Ni) that can be coordinated is shown in the column “Mixing Amount” of Table 1. The pH of the water phase was 3.1.

12 mL of a PC-88A solution as the extractant solution was added to the water phase and was stirred using a stirrer chip at 25° C. for 30 minutes. In this case, a mixing amount (unit: equivalent) of the extractant (C) with respect to a total content of the metal ions (here, Co and Ni) that can be coordinated is shown in the column “Mixing Amount” of Table 1. Next, ammonia water (manufactured by FUJIFILM Wako Pure Chemicals Corporation, concentration: 28 to 30 mass %) was added to adjust the pH of the mixed solution to 4.3. Further, the solution was stirred at 25° C. for 30 minutes, and was left to stand at the same temperature for 1 hour. After verifying that the solution was separated into two layers of the organic phase (oil phase) and the water phase, the separated water phase was extracted, and the separation and recovery of the metal ions was performed.

### Comparative Examples 1 and 2

The separation and recovery of the metal ions according to Comparative Examples 1 and 2 was performed using the same method as that of Example 1, except that 1.5 mL of ultrapure water was added instead of the organic compound aqueous solution, the metal ion-containing aqueous solution and the extractant solution were changed to a combination shown in Table 1, and the pH during the mixing of the water phase and the oil phase was set to a value shown in the column “pH during Mixing” of Table 1 for mixing and standing.

### Comparative Example 3

### <Preparation of Metal Ion-Containing Aqueous Solution>

15.5 g of cobalt (II) sulfate heptahydrate (manufactured by FUJIFILM Wako Pure Chemicals Corporation) and 15.5 g of nickel (II) sulfate heptahydrate (manufactured by FUJIFILM Wako Pure Chemicals Corporation) were added to a 1L measuring flask, were diluted with ultrapure water, and were stirred and dissolved at 30° C. As a result, a metal ion-containing aqueous solution including two kinds of metal ions was prepared.

### <Preparation of Extractant Solution>

PC-88A as the extractant (C) was added to a 100 mL measuring flask, and the solution was diluted using kerosine at room temperature. As a result, a PC-88A solution (concentration: 310 mM) was prepared as an extractant solution.

### <Separation Recovery of Metal Ions>

1.5 mL of ultrapure water with respect to 10 mL of the prepared metal ion-containing aqueous solution was added to a 30 mL vial tube and was stirred at 25° C. for 10 minutes. This way, a water phase including 2 kinds of metal ions (A) was prepared. The pH of the water phase was 3.2.

12 mL of a PC-88A solution as the extractant solution was added to the water phase and was stirred using a stirrer chip at 25° C. for 30 minutes. In this case, a mixing amount (unit: equivalent) of the extractant (C) with respect to a total content of the metal ions (here, Co and Ni) that can be coordinated is shown in the column “Mixing Amount” of Table 1. Next, ammonia water (manufactured by FUJIFILM Wako Pure Chemicals Corporation, concentration: 28 to 30 mass %) was added to adjust the pH of the mixed solution to a value shown in the column “pH during Mixing” of Table 1. Further, the solution was stirred at 25° C. for 30 minutes, and was left to stand at the same temperature for 1 hour. After verifying that the solution was separated into two layers of the organic phase (oil phase) and the water phase, the separated water phase was extracted, and the separation and recovery of the metal ions was performed.

Regarding each of the water phases obtained Examples and Comparative Examples (each of the used water phases and each of the extracted water phases), the pH was measured using a pH meter (SK-620 pH II, manufactured by SATOTECH), and each of the contents of dissolved metal ions was determined using an inductively coupled plasma-optical emission spectrometer (ICP-OES) (Optima 7300 D (trade name), manufactured by Perkin Elmer Co., Ltd.). Measured values of the pH of each of the water phases used in Examples and Comparative Examples and the content of dissolved metal ions in each of the water phases are shown in the column “Water Phase pH” and the column “Metal Ion Concentration (ppm)” of Table 1.

In addition, in Examples and Comparative Examples, the results measured using the same method as that of the pH during the mixing of the water phase and the oil phase are shown in the column “pH during Mixing”. Further, the mixing amounts of the organic compound (B) and the extractant (C) with respect to the total content of the metal ions that can be coordinated are shown in the columns “Mixing Amounts” of Table 1. In Table 1, the unit of the mixing amount is equivalent but is not shown.

TABLE 1

| | Metal Ion Concentration in Water | | | | | Metal Ion Concentration in Water | | | | |
|---|---|---|---|---|---|---|---|---|---|---|
| | Phase before Extraction (ppm) | | | | | Phase after Extraction (ppm) | | | | |
| No. | Fe | Zn | Co | Ni | In | Fe | Zn | Co | Ni | In |
| Example 1 | 15,000 | 15,000 | 15,000 | 15,000 | 15,000 | 11,000 | 19 | 15,000 | 15,000 | 15,000 |
| Example 2 | 15,000 | 15,000 | 15,000 | 15,000 | 15,000 | <1 | 11,000 | 15,000 | 15,000 | 15,000 |
| Example 3 | 15,000 | 15,000 | 15,000 | 15,000 | 15,000 | <1 | 13,000 | 15,000 | 15,000 | 15,000 |
| Example 4 | 15,000 | 15,000 | 15,000 | 15,000 | 15,000 | <1 | 14,800 | 15,000 | 15,000 | 15,000 |
| Example 5 | 0 | 15,000 | 15,000 | 15,000 | 15,000 | 0 | <1 | 15,000 | 15,000 | 15,000 |
| Example 6 | 0 | 0 | 15,000 | 15,000 | 0 | 0 | 0 | <1 | 12,000 | 0 |
| Example 7 | 0 | 15,000 | 0 | 0 | 15,000 | 0 | 240 | 0 | 0 | 15,000 |
| Example 8 | 15,000 | 15,000 | 0 | 0 | 15,000 | 160 | 15,000 | 0 | 0 | 15,000 |
| Example 9 | 0 | 0 | 15,000 | 15,000 | 0 | 0 | 0 | <1 | 14,000 | 0 |
| Example 10 | 0 | 0 | 15,000 | 15,000 | 0 | 0 | 0 | 140 | 11,000 | 0 |
| Example 11 | 0 | 0 | 15,000 | 15,000 | 0 | 0 | 0 | <1 | 12,000 | 0 |
| Example 12 | 0 | 0 | 15,000 | 15,000 | 0 | 0 | 0 | <1 | 12,000 | 0 |
| Example 13 | 0 | 0 | 15,000 | 15,000 | 0 | 0 | 0 | 10,000 | 650 | 0 |
| Example 14 | 0 | 0 | 15,000 | 15,000 | 0 | 0 | 0 | <1 | 10,000 | 0 |
| Example 15 | 0 | 0 | 15,000 | 15,000 | 0 | 0 | 0 | 3,000 | 11,000 | 0 |
| Comparative | 15,000 | 15,000 | 15,000 | 15,000 | 15,000 | 80 | 12 | 15,000 | 15,000 | 15,000 |
| Example 1 | | | | | | | | | | |
| Comparative | 0 | 0 | 15,000 | 15,000 | 0 | 0 | 0 | <1 | 5,500 | 0 |
| Example 2 | | | | | | | | | | |
| Comparative | 0 | 0 | 15,000 | 15,000 | 0 | 0 | 0 | <1 | 6,200 | 0 |
| Example 3 | | | | | | | | | | |

| | | | | Organic | | | | |
|---|---|---|---|---|---|---|---|---|
| | Extracted | Water | pH | Compound (B) | | Extractant (C) | | |
| | Metal | Phase | during | | Mixing | | | Mixing |
| | Ions | pH | Mixing | | Amount | | pKa | Amount |
| Example 1 | Zn, Fe | 4.1 | 2.8 | Thiourea | 1.4 | TEHA | 9.8 | 3.5 |
| Example 2 | Fe, Zn | 2.5 | 0.5 | Thiourea | 1.4 | PC-88A | 4.6 | 3.5 |
| Example 3 | Fe, Zn | 2.0 | 0.6 | EDA | 1.4 | PC-88A | 4.6 | 3.5 |
| Example 4 | Fe, Zn | 2.0 | 0.5 | EDTA | 1.4 | PC-88A | 4.6 | 3.5 |
| Example 5 | Zn | 3.5 | 2.6 | EDTA | 1.4 | PC-88A | 4.6 | 3.5 |
| Example 6 | Co, Ni | 2.5 | 4.0 | EDTA | 1.4 | PC-88A | 4.6 | 3.5 |
| Example 7 | Zn | 2.0 | 3.0 | EDTA | 1.4 | PC-88A | 4.6 | 3.5 |
| Example 8 | Fe | 0.9 | 0.2 | EDTA | 1.4 | PC-88A | 4.6 | 3.5 |
| Example 9 | Co, Ni | 3.1 | 4.1 | EDTA | 1.4 | PC-88A | 4.6 | 3.5 |
| Example 10 | Co, Ni | 3.5 | 4.5 | EDTA | 1.4 | VA-10 | 7.9 | 3.5 |
| Example 11 | Co, Ni | 3.2 | 4.2 | EDTA-OH | 1.4 | PC-88A | 4.6 | 3.5 |
| Example 12 | Co, Ni | 3.2 | 4.2 | DPTA | 1.4 | PC-88A | 4.6 | 3.5 |
| Example 13 | Ni, Co | 6.2 | 7.0 | EDDA | 1.4 | PC-88A | 4.6 | 3.5 |
| Example 14 | Co, Ni | 3.0 | 3.9 | NTA | 1.4 | PC-88A | 4.6 | 3.5 |
| Example 15 | Co, Ni | 3.1 | 4.3 | EDTA | 1.4 | PC-88A | 4.6 | 3.5 |
| Comparative | Zn, Fe | 4.0 | 2.6 | — | — | TEHA | 9.8 | 3.5 |
| Example 1 | | | | | | | | |
| Comparative | Co, Ni | 3.2 | 4.2 | — | — | PC-88A | 4.6 | 3.5 |
| Example 2 | | | | | | | | |
| Comparative | Co, Ni | 3.2 | 4.2 | — | — | PC-88A | 4.6 | 3.5 |
| Example 3 | | | | | | | | |

### <Notes in Table>

Thiourea: manufactured by FUJIFILM Wako Pure Chemicals Corporation
EDA: ethylenediamine (manufactured by Tokyo Chemical Industry Co., Ltd.)
TEHA: tris(2-ethylhexyl)amine shown below (manufactured by Tokyo Chemical Industry Co., Ltd.)
PC-88A: mono-2-ethylhexyl (2-Ethylhexyl)phosphonate shown below (manufactured by Tokyo Chemical Industry Co., Ltd.)
VA-10: Versatic acid 10 (manufactured by Hexion Specialty Chemicals, Inc.)

In the table, compounds represented by reference numerals are shown below. As these compounds, commercially available products were used.

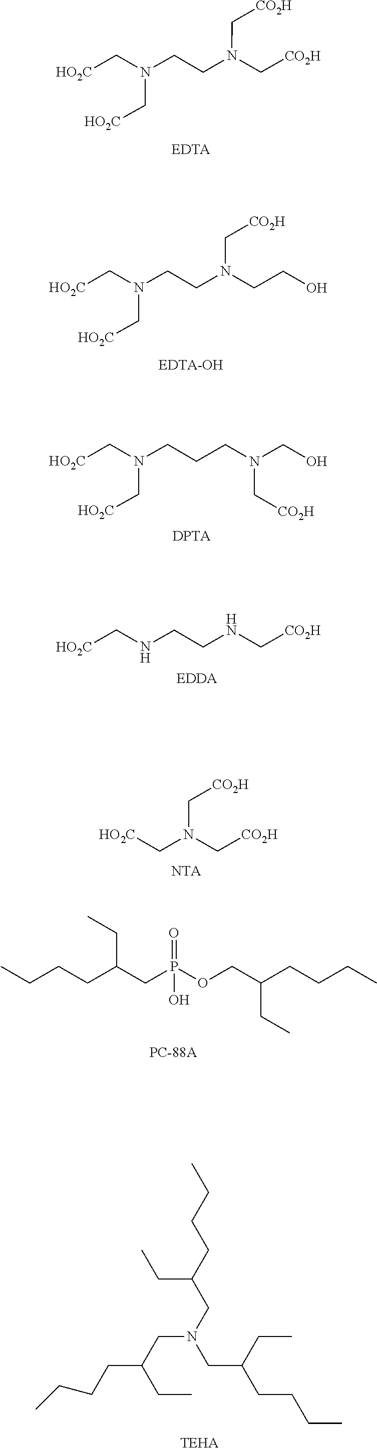

The following can be seen from the results of “Metal Ion Concentration (ppm) in Water Phase before Extraction” and “Metal Ion Concentration (ppm) in Water Phase after Extraction” shown in Table 1.

In Comparative Example 1 where the organic compound (B) was not used in combination with the extractant (C) in the separation and recovery of the metal ions from the water phase, the concentration of extracted Zn was about 1.0 times the concentration of Fe, and Fe and Zn were not able to be separated with high separability. Likewise, in Comparative Example 2, the concentration of extracted Co was about 1.6 times the concentration of Ni, and Co and Ni were not able to be separated with high separability. In Comparative Example 3 where ammonia was used in combination with the extractant (C) instead of the organic compound (B), although Co and Ni were able to be recovered, the concentration of extracted Co was about 1.7 times the concentration of Ni, and the separability of Co and Ni was not sufficient.

On the other hand, in Examples 1 to 4 where the extractant (C) and the organic compound (B) were used in combination in the separation and recovery of the metal ions from the water phase, Fe and Zn were able to be separated from the other metal ions with high separability, the concentration of one of the extracted Zn or Fe was about 3.7 times or more of the concentration of the other one, and the separability of Fe and Zn was also high. Likewise, in Examples 5, 7, and 8, only Zn or only Fe was able to be separated and recovered from the other metal ions with high separability. Further, in Examples 6 and 9 to 14 where the separation and recovery from the water phase including two kinds of metal ions of Co and Ni was performed using a combination of the extractant (C) and the organic compound (B), the concentration of one of the extracted Co or Ni was about 2.9 times or more of the concentration of the other one, and one of Co or Ni was able to be separated and recovered from the other one of Co or Ni with high separability.

It can be seen from the above results that the metal ions extracted to the oil phase with high separability can be separated and recovered without deterioration of the high separability by stripping the oil phase obtained in each of Examples using a typical method under typical conditions.

The present invention has been described using the embodiments. However, unless specified otherwise, any of the details of the above description is not intended to limit the present invention and can be construed in a broad sense within a range not departing from the concept and scope of the present invention disclosed in the accompanying claims.

What is claimed is:

1. A separation recovery method of metal ions, the separation recovery method comprising:
mixing a water phase including two or more kinds of metal ions (A) and an organic compound (B) with an oil phase including an extractant (C) to move ions of metal elements belonging to Group 8 to Group 12 in a periodic table of elements from the water phase to the oil phase, the two or more kinds of metal ions (A) including ions of metal elements belonging to Group 3 to Group 16 in the periodic table of elements, and the organic compound (B) having a coordinating functional group coordinated to at least one kind of metal ions among the two or more kinds of metal ions (A).

2. The separation recovery method according to claim 1,
wherein the extractant (C) is an acidic extractant.

3. The separation recovery method according to claim 1,
wherein the extractant (C) is represented by Formula (I) or Formula (II), 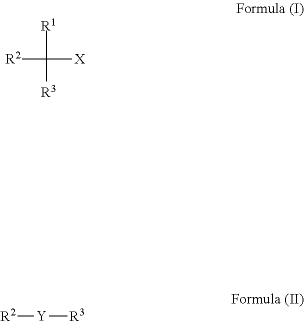
in Formula (I) and Formula (II),
R¹ represents an alkyl group, R² and R³ represent an organic group and may be the same as or different from each other,
X represents a carboxy group, a sulfonate group, a sulfinate group, a phosphate group, a phosphonate group, or an oxime group, and
Y represents a phosphinate group, a phosphonate group, a phosphate group, a sulfonate group, or an oxime group.

4. The separation recovery method according to claim 1,
wherein the organic compound (B) is a chelating agent.

5. The separation recovery method according to claim 1,
wherein the organic compound (B) includes at least one element of N, O, S, or P in a molecular structure of the organic compound (B).

6. The separation recovery method according to claim 1,
wherein the organic compound (B) is represented by Formula (III), 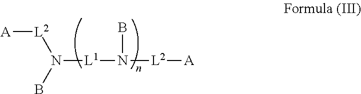
in Formula (III),
L¹ represents a divalent organic group, L² represents a single bond or a divalent organic group, and two L²'s may be different from each other,
A represents a hydroxy group, an amino group, a carboxy group, a sulfonate group, a sulfinate group, a phosphate group, phosphonic acid, a cyano group, a carbamoyl group, or a thiol group, and two A's may be different from each other,
B represents a monovalent organic group or a hydrogen atom and, in a case where a plurality of B's are included, the plurality of B's may be different from each other, and n represents an integer of 0 to 8.

7. The separation recovery method according to claim 1,
wherein the two or more kinds of metal ions (A) include at least one transition metal element.

8. The separation recovery method according to claim 1,
wherein the metal ions to be moved to the oil phase are ions of two metal elements belonging to different groups among ions of metal elements belonging to Group 8 to Group 11 in the periodic table of elements.

9. The separation recovery method according to claim 1,
wherein the two or more kinds of metal ions (A) are a metal recovery from a waste battery.

10. A two-phase separated fluid,
wherein a water phase including two or more kinds of metal ions (A) and an organic compound (B) and an oil phase including an extractant (C) are phase-separated and present in a state where the water phase and the oil phase are in contact with each other, the two or more kinds of metal ions (A) including ions of metal elements belonging to Group 3 to Group 16 in a periodic table of elements, and the organic compound (B) having a coordinating functional group coordinated to at least one kind of metal ions among the two or more kinds of metal ions (A), and
metal ions where the extractant (C) is coordinate-bonded to metal elements belonging to Group 8 to Group 12 in the periodic table of elements among the two or more kinds of metal ions (A) are present in the oil phase.

11. The two-phase separated fluid according to claim 10,
wherein the extractant (C) is an acidic extractant.

12. The two-phase separated fluid according to claim 10,
wherein the extractant (C) is represented by Formula (I) or Formula (II), 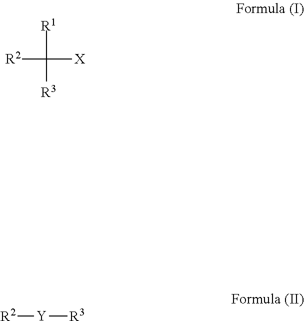
in Formula (I) and Formula (II),
R¹ represents an alkyl group, R² and R³ represent an organic group and may be the same as or different from each other,
X represents a carboxy group, a sulfonate group, a sulfinate group, a phosphate group, a phosphonate group, or an oxime group, and
Y represents a phosphinate group, a phosphonate group, a phosphate group, a sulfonate group, or an oxime group.

13. The two-phase separated fluid according to claim 10,
wherein the organic compound (B) is a chelating agent.

14. The two-phase separated fluid according to claim 10,
wherein the organic compound (B) includes at least one element of N, O, S, or P in a molecular structure of the organic compound (B).

15. The two-phase separated fluid according to claim 10,
wherein the organic compound (B) is represented by Formula (III), 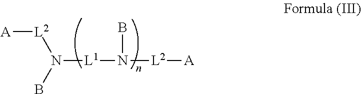
in Formula (III),
L¹ represents a divalent organic group, L² represents a single bond or a divalent organic group, and two L²'s may be different from each other,
A represents a hydroxy group, an amino group, a carboxy group, a sulfonate group, a sulfinate group, a phosphate group, phosphonic acid, a cyano group, a carbamoyl group, or a thiol group, and two A's may be different from each other,
B represents a monovalent organic group or a hydrogen atom and, in a case where a plurality of B's are included, the plurality of B's may be different from each other, and
n represents an integer of 0 to 8.

16. The two-phase separated fluid according to claim 10,
wherein the two or more kinds of metal ions (A) include at least one transition metal element.

17. The two-phase separated fluid according to claim 10,
wherein the metal ions present in the oil phase are ions of two metal elements belonging to different groups among ions of metal elements belonging to Group 8 to Group 11 in the periodic table of elements.
